(19) United States
(12) Patent Application Publication
(10) Pub. No.: US 2003/0199439 A1
(43) Pub. Date: Oct. 23, 2003

(54) Compositions of alpha3beta4 receptor antagonists and opioid agonist analgesics

(76) Inventor: David Lew Simon, Mansfield Center, CT (US)

Correspondence Address: David L. Simon, M.D., P.O. Box 618, Mansfield Center, CT, 06250, US

(21) Appl. No.: 10/127,359

(22) Filed: Apr. 22, 2002

(51) Int. Cl. A61K 38/33; A61K 31/7052; A61K 31/485; A61K 31/445; A61K 31/192

(52) U.S. Cl. 514/12; 514/282; 514/317; 514/23; 514/161; 514/570

(57) ABSTRACT

A pharmaceutical composition comprising an opioid agonist analgesic and an α3β4 nicotinic receptor antagonist effective to separate the brain-derived wanting of the opioid from the analgesic or anti-diarrhea effect of the opioid agonist.

### BACKGROUND

1. Field of Invention

This invention relates to pharmaceutical compositions, specifically to those containing opioid agonist analgesics as at least one component of the composition.

2. Description of the Prior Art

Opioid agonist analgesics have long been a cornerstone of pharmaceutical management of pain and other medical maladies such as loose stool or diarrhea. However, use of opioid agonist analgesics may be accompanied by feeling euphoria as a reaction apart from relief of pain, or may be accompanied by other pharmaceutical effects as to create a wanting of the opioid agonist analgesic as an issue separate and distinct from the issue of pain relief. It is undesirable for a human patient to want to be administered an opioid agonist analgesic for reasons other than relief of pain or prescribed treatment of licit medical maladies such as loose stool. Such a wanting could result in the opioid agonist analgesic being administered in quantities greater than that required to treat pain and other licit medical maladies, which would result in waste of opioid agonist analgesic, and an increase in spending for opioid agonist analgesics. This is of great societal significance in managing the allocation of scarce resources available in the treating health care system in general. Any wastage of money on a pharmaceutical or medication results in less money available for other needed resources, be they other medications or health care services. In and of itself, a decrease in wanting of opioids apart from pain relief and other licit uses (hereafter “any licit use”) would be of great of great utility, whether it be in an opioid naive individual (i.e., one that has not been previously exposed to opioid analgesics) or an individually chronically exposed to opioid agonist analgesics (e.g., a chronic pain patient, as one who is long suffering from malignant or cancer-related pain).

There have been attempts to reduce the effective amount of opioid agonist analgesic for any licit use. Such attempts have included the co-administration of opioids with NMDA-receptor antagonists or relatively low doses of opioid antagonist. These methods, if effective, could theoretically serve the desired purpose of reducing wastage of opioids, however these methods have not been demonstrated to decrease the wanting of the opioid apart from any licit use, and in fact, could theoretically potentiate the opioid agonist effect to possibly increase the wanting desire of the opioid agonist analgesic, which would have the opposite of the desired effect to decrease wastage and optimize management of scare health care resources.

Crain et al teach that very small doses of opioid antagonists may potentiate the analgesic effect of opioid agonist analgesics (U.S. Pat. Nos. 5,580,876 and 5,767,125). Crain does not mention nicotinic receptors of any sort, including α3β4 nicotinic receptors or their antagonists. Crain also does not teach decreasing the wanting desire of opioid analgesics apart from any licit use.

The present author teaches that a pharmaceutical composition comprising nalmefene and an opioid agonist analgesic may optimize dopamine homeostasis while dissuading a human from abusing the opioid agonist analgesic (U.S. Pat. No. 6,103,258, hereafter “'258”). This invention, however, does not utilize nicotinic receptors, and it has a ceiling effect for any given combination of nalmefene and opioid. Further, specific drug combinations in varying ratios of nalmefene to opioid must be formulated to effectively deliver therapeutic doses of a particular. opioid agonist analgesic.

Mayer, et al teach that NMDA (N-methyl-D-aspartate) receptor antagonists such as dextromethorphan or dextrophan may be combined with opioid agonist analgesics for the prevention of opioid tolerance (U.S. Pat. No. 5,654,281). However, this may make the opioid agonist effectively more potent, and Mayer does not teach that this invention will decrease the wanting or desire for being administered opioids apart from the effect of any licit use.

Caruso teaches that NMDA receptor antagonists administered with narcotic agonist/antagonists increase the analgesic effect of the agonist/antagonist (U.S. Pat. No. 6,007,841). Again, this may render the opioid agonist more potent and does not speak to decreasing the wanting of the opioid apart from the effect of any licit use. Caruso makes no mention of α3β4 nicotinic receptors or its antagonists.

Palermo, et al (U.S. Pat. No. 6,228,863) and Kaiko, et al (U.S. Pat. No. 6,277,384) teaching compositions for oral administration containing opioid agonist analgesics and opioid antagonists in varying amounts depending upon the particular opioid agonist and antagonist used. These formulations, however, have the potential to produce precipitated abstinence syndrome in susceptible individuals, unlike the present invention. Unintentional precipitated abstinence syndrome (“withdrawal”) can have serious deleterious effects on humans, such as precipitation of catecholamine release, exaggerated stress response and myocardial ischemia. Unmonitored, as may occur with an unintentional withdrawal, this could be life threatening.

Smith, et al teach that a kappa-2 opioid receptor agonist may be combined with a mu opioid receptor agonist such that relatively low sub-therapeutic doses of each may result in therapeutic analgesia (U.S. Pat. No. 6,310,072). However, Smith does not teach that this invention reduces wanting for the combined drug combination apart from any licit use.

Hamann teaches a composition comprising mecamylamine and naltrexone for the treatment of pain, where mecamylamine is a nicotinic receptor antagonist (U.S. Pat. No. 6,153,621, hereafter “'621). It is doubtful and highly unlikely that the invention claimed by Hamann could produce comparable analgesia as an opioid agonist analgesic as is contained in the present invention. Though Hamann does make mention in one broad stroke of “using nicotinic, opioid, adrenergic and/or seratonergic antagonists with agonists,” he describes the “agonists” used as both opioid and nicotinic agonists: “Suitable opioid agonists . . . include . . . morphine,” etc., and “Suitable nicotinic agonists include . . . (−)-nicotine,” etc.” It is not at all clear that Hamann means to teach a combination of an opioid agonist analgesic combined with a nicotine receptor antagonist. In fact, his accepted claims speak to just the opposite, i.e., his first and subsequent claims claim an opioid antagonist (e.g. naltrexone) in combination with a nicotinic antagonist. Further, '621 does not purport to teach the decrease for wanting of an opioid agonist analgesic, as is taught in the present invention.

Glick, et al teach the manufacture of ibogaine congeners, including α3β4 nicotinic receptor antagonists (U.S. Pat. No. 6,211,360, hereafter “'360”). '360 claims various methods “of treating a subject's addiction” comprising the administration of the claimed manufactured ibogaine congener for various addictions “wherein the addictive substance is an opiate.” Glick does not teach administering α3β4 receptor antagonists in combination with opioid agonist analgesics for any licit use in those that are not addicted, as does the present invention. In fact, '360 does not teach a composition combining an opioid agonist analgesic with a α3β4 nicotinic receptor antagonist at all. '360 is specifically modeled after Lotsof (U.S. Pat. Nos. 4,499,096; 4,587,243; 5,152,994) where “a single oral treatment with ibogaine or its salts” is administered to treat a particular addiction. Glick quite clearly describes the method of '360 as “administering to the addicted subject an effective amount of a compound [ibogaine congener] having the . . . formula” without co-administration of the drug to which the subject is addicted. Thus, even to someone as expert and skilled in the art of Glick and colleagues, the present invention was not appreciated.

Though stereospecificity of α3β4 receptors for ibogamine, ibogaine and their congeners may not (or may) be clinically relevant, the sterospecificity of opioid receptors for ibogamine, ibogaine and their congeners does seem to be important. For instance, the (+)-enantiomer of 18-MC has a 10-fold higher affinity for mu- and delta-opioid receptors than does the corresponding (−)-enantiomer (Bioorganic & Medicinal Chemistry Letters 10 (2000) 473-476). '360 specifically states that the '360 ‘invention includes compounds . . . without regard to the stereochemistry . . . . ” This is further evidence that '360 did not encompass, consider or teach the present invention that is a composition containing both opioids acting at mu- and delta-opioid receptors and ibogamine, ibogaine or their congeners that are α3β4 nicotinic receptor antagonists. It cannot be presumed otherwise that a single composition containing both opioid agonists and compounds whose stereoisomer orientation alters competition with binding at opioid receptors would not fit the definition of an invention that is “without regard to the stereochemistry.”

Glick, Maisonneuve, Kitchen and Fleck (European Journal of Pharmacology 438 (2002) 99-105) describe “antagonism of α3β4 nicotinic receptors as a strategy to reduce opioid and stimulant self-administration.” The prototypical α3β4-antagonist is 18-methoxycoronaridine (“18-MC”). They teach that 18-MC is a “potential treatment for multiple forms of drug abuse” and they do not distinguish the utility of decreasing the wanting of opioid agonist analgesics specifically, as does the present invention. Further, nowhere is it suggested to compound or formulate a pharmaceutical composition containing both opioid agonist analgesic and α3β4 nicotinic receptor antagonist. This article references other works by the same authors where 18-MC is specifically administered as a pre-treatment 19 hours prior to the administration of morphine (European Journal of Pharmacology 383 (1999) 15-21). That 18-MC is administered as a separate and distinct pre-treatment, not possibly administered as a single composition containing both 18-MC and active drug (where “active drug” is meant to mean opioid agonist analgesic or metamphetamine) is again evidence by an article written by Szumlinski, Haskew, Balogun, Maisonneuve and Glick (Pharmacology, Biochemistry and Behavior 69 (2001) 485-491) that again describes the pretreatment with ibogaine or its congener 19 hours prior to administration of active drug, which in this case was methamphetamine. Even in the article titled “The potential ant-addictive agent, 18-methoxycoronaridine, blocks the sensitized locomotor and dopamine responses produced by repeated morphine treatment” authored by Szumlinski, Maisonneuve and Glick (Brain Research 864 (2000) 13-23), 18-MC is always administered as a pretreatment 19 hours prior to morphine administration, again giving no evidence or suggestion that these authors teach a single pharmaceutical composition comprising both α3β4 nicotinic receptor antagonist and opioid agonist analgesic, as is taught in the present invention. In fact, the authors concluded in this article that “it appeared that 18-MC pretreatment [emphasis added] blocked the expression of sensitization in rats sensitized by previous morphine exposure” (without previous co-administration of 18-MC).

In Opioids In Pain Control: Basic and Clinical Aspects (ISBN 0 521 62269 7), a diagram labeled FIG. 6.3 is shown, marked herein as FIG. 1. FIG. 1 demonstrates that dopamine is increased in striato-pallidal regions of the brain with administration of a prototypical opioid, as is morphine. Other explanations of opioid-induced increase in brain dopamine is offered by Garcia and Harlan in The Neurobiology of Opiates (ISBN 0-8493-7932-6): it explains beta-endorphin infusion into the nucleus accumbens induces dopamine release there, and morphine infusion into the ventral tegmental area induces dopamine release there. In the same book (ISBN 0 521 62269 7), Unterwald and Kornetsky state as a theory that “opiates activate opiate receptors located in the ventral tegmental area, which in turn stimulate dopaminergic activity in the mesolimbic system, which mediates reinforcement.” It is dopamine increase in response to opioids in the mesolimbic system that is responsible for wanting the drug again apart from any licit use of the opioid agonist analgesic. Antagonism of α3β4 nicotinic receptors indirectly alter dopamine in the nucleus accumbens and ventral tegmental area by communication via the habenulointerpedunclular pathway as explained by Glick (Ibid). Any licit use of an opioid agonist analgesic is mediated via opioid receptors, e.g. analgesia, which may occur independent of interactions involving wanting of the drug. It would be of very great utility to separate the wanting or reinforcing effect of being administered an opioid agonist analgesic from any licit use of the opioid agonist as taught in the present invention.

There are at least three general mechanisms by which opioid agonist analgesics effect the relief of pain: 1) supraspinal mechanisms of opioid analgesia; 2) spinal mechanisms of opioid analgesia; and, 3) peripheral mechanisms of opioid analgesia.

Supraspinal opioid analgesia occurs primarily in areas of the brain apart from the pleasure-reward center in the mesolimbic system, such as the midbrain periaqueductal grey (“PAG) area, and rostral ventromedial medulla (“RVM”). The anatomic organization of this pain modulating network is shown schematically in FIG. 2 (labeled FIG. 3.1 from Opioids In Pain Control: Basic and Clinical Aspects (ISBN 0 521 62269 7). Opioid actions within the RVM are mediated by GABA as a major neurotransmitter. Yaksh and Rudy demonstrated that if opioid actions are blocked in only the spinal cord (and not the brain) by an opioid antagonist, opioid-like effects of systemically administered morphine is completely blocked. Though this may be due to complex interactions among and between spinal and supraspinal sites, this demonstrates that any licit use of an opioid agonist analgesic can be separated from the wanting or reinforcing effects of opioid administration that occurs primarily in an anatomical location within the brain (the “pleasure-reward center”). Pain is mediated mostly by mu receptors in the spinal cord, and diarrhea is mediated by opioid receptors located within the gastrointestinal track. Further many actions of analgesia and tolerance involving both opioid and NMDA receptors are mediated in the spinal cord and peripheral sites in the body, away from the actions of α3β4 receptor antagonists in the pleasure-reward center of the brain (see for example, Science Jun. 25 , 1976 ;192(4246):1357-8 and Anesthesiology May 1996;84(5):1177-88 and Anesthesiology October 1996;85(4):808-16 and Neurosci Lett Jan. 20, 1992;135(1):67-70 and J Pain Symptom Manage August 1992;7(6):356-61 and J Pharmacol Exp Ther April 1999;289(1):494-502).

Opioid agonist analgesics prescribed to reduce or alleviate pain working at the level of the spinal cord apart and away from the pleasure-reward center of the brain work by way of interaction of multiple neurotransmitters. Primarily, neurotransmitters in the spinal cord are in the dorsal horn of the spinal cord and include excitatory amino acids and certain neuropeptides (e.g. substance P and calcitonin gene-related peptide also known as CGRP), cholecystokinin (“CCK”), and Met-enkephalin. These are not known to be affected in the dorsal horn of the spinal cord by α3β4 nicotinic receptor antagonists.

FIG. 3 (labeled FIG. 4.3 in Opioids In Pain Control: Basic and Clinical Aspects (ISBN 0 521 62269 7) is a schematic representation of interactions among and between neurotransmitters in the spinal cord.

From Substance Abuse: A Comprehensive Textbook (ISBN 0-683-18179-3), it is stated “opiate-induced enhancement of the firing of reward-relevant mesotelencephalic dopamine neurons is well established. Mesolimbic dopamine neurons originating in the ventral tegmental area and projecting to the nucleus accumbens are preferentially sensitive to this opiate-induced activation.” The homeostatic control of dopamine in this area of the brain is under opposing tonic control by mu- and kappa-opioid receptors as taught in '258. Nevertheless, “the action of mu-opioid receptor agonists on the firing rate of mesotelencephalic dopamine neurons in reward-relevant brain loci is primarily an activating one” (ISBN 0-683-18179-3). For all intent and purpose, opioid agonist analgesics administered to treat pain are essentially mu-opioid agonists. “Nicotine also acutely activates mesotelencephalic dopamine neurons. The same range of doses [of nicotine] was more than three times as effective on mesolimbic dopamine neurons as on mesostriatal dopamine neurons, and all nicotine-induced activation of dopamine neurons was prevented or reversed by intravenous mecamylamine” (ISBN 0-683-18179-3).

### OBJECTS AND ADVANTAGES

Accordingly, besides the objects and advantages of formulating or compounding a pharmaceutical composition containing both an opioid agonist analgesic and a α3β4 receptor antagonist described in my invention, above, several objects and advantages of the present invention are:

(a) to allow an opioid to be administered to a human effective to relieve pain while simultaneously not allowing for increased dopamine in regions of the brain that would effect wanting of an opioid or euphoria, in a single pharmaceutical composition;

(b) to decrease the tendency of a human to self-administer opioid agonist analgesics for reasons other than any licit use;

(c) to treat pain without affecting mood as an opioid analgesic in absence of a α3β4 nicotinic receptor antagonist would;

(d) in absence of pain, to decrease the tendency of a human to self-administer an opioid agonist analgesic;

Further objects advantages of the present invention will become apparent from a consideration of the ensuing description and figures.

### DRAWING FIGURES

FIG. 1 demonstrates striatal dopamine increases with use of the opioid agonist morphine.

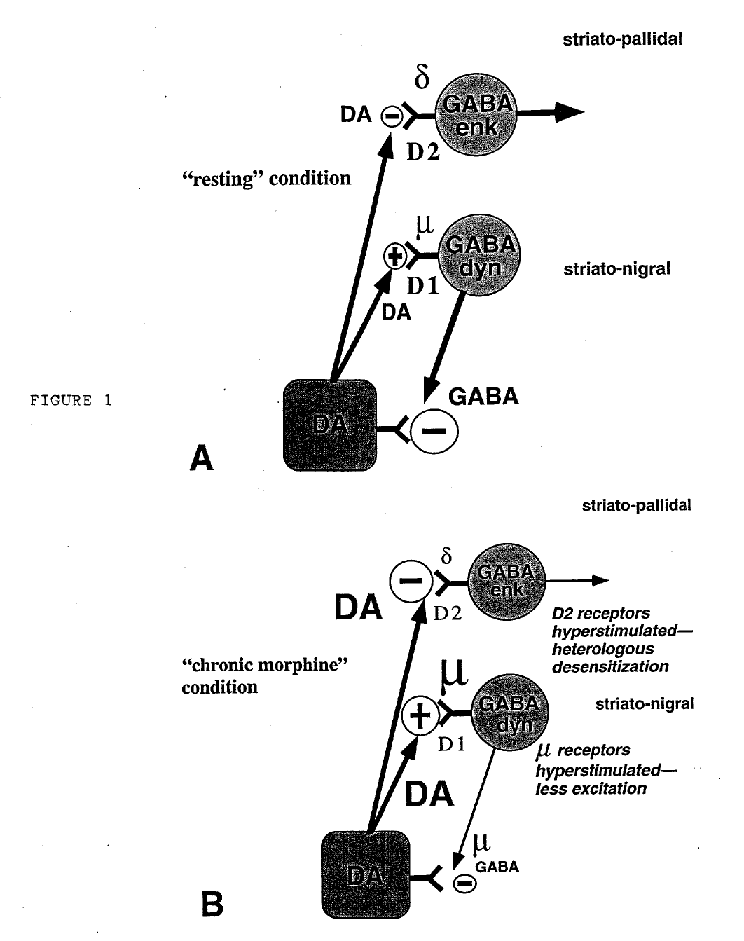

FIG. 2 demonstrates that areas of the brain other than those regions related to mesolimbic pleasure, reward and wanting are central to modulating pain mediated by opioid receptors in the brain.

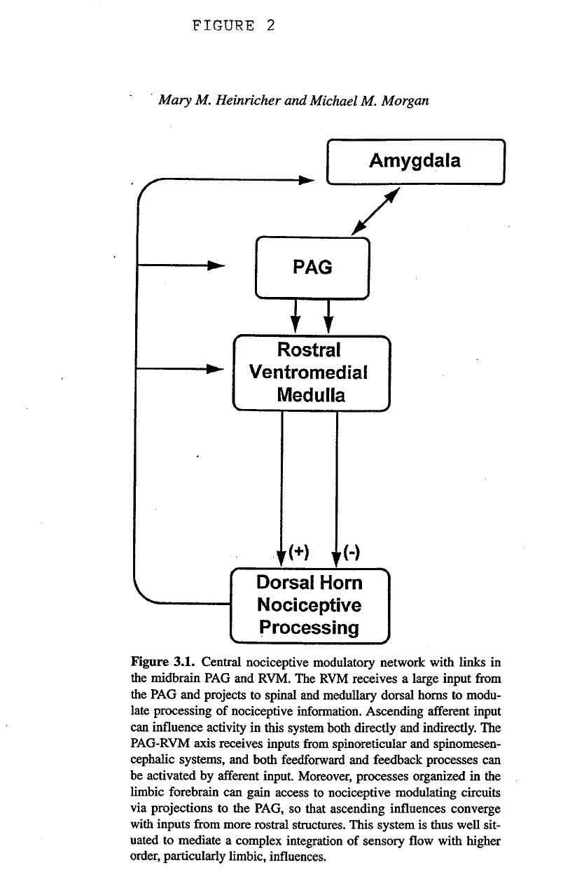

FIG. 3 demonstrates that neurotransmitters other than dopamine are effective in transmission of pain mediated by opioid receptors in the spinal cord.

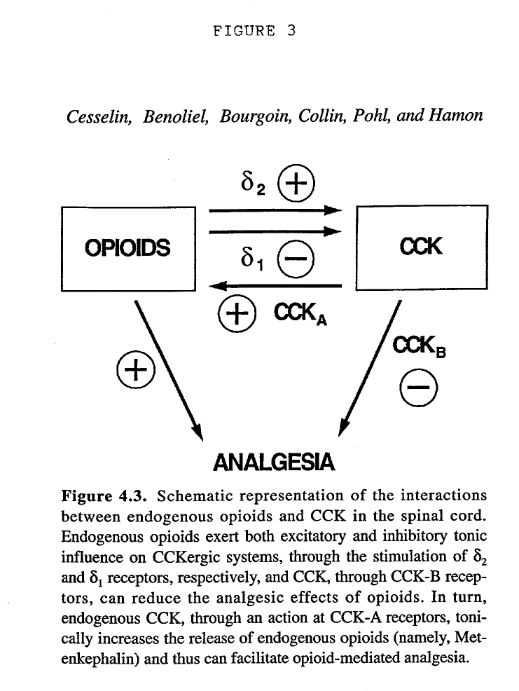

### DESCRIPTION OF THE INVENTION

The present invention consists of an amount of an opioid agonist analgesic effective to produce a positive physiologic response for any licit use of the opioid analgesic, and an amount of an α3β4 (“alpha-three-beta-four”) nicotinic receptor antagonist effective to inhibit, antagonize, prevent or diminish dopaminergic effects within the area of the brain responsible for pleasure, reward or wanting, in the same pharmaceutical composition, and a suitable carrier therefore.

In one embodiment of the invention, the α3β4 nicotinic receptor antagonist is 18-methoxycoronaridine (hereafter, “18-MC”). However, any number of suitable α3β4 nicotinic receptor antagonists may be used. Other such suitable antagonists include mecamylamine, dextromethorphan and dextrophan. In light of the present invention, the development of other α3β4 nicotinic receptor antagonists may occur that may also be suitable for the invention. The method by which one may test a drug for α3β4 nicotinic receptor antagonism is previously described in the scientific literature and would be known to one skilled in that art. A list of possible candidates for being an α3β4 nicotinic receptor antagonist in addition to the stated known α3β4 nicotinic receptor antagonists include: 18-hydroxycoronaridine; 18-hydroxyvoacangine; 18-hydroxyconopharyngine; 16-ethoxycarbonyl-18-hydroxyibogamine; 16-ethoxycarbonyl-18-hydroxyibogaine; 16-ethoxycarbonyl-18-hydroxyibogaline; 16-hydroxymethyl-18-hydroxyibogamine; 16-hydroxymethyl-18-hydroxyibogaine; 16-hydroxymethyl-18-hydroxyibogaline; 18-methoxyvoacangine; 18-methoxyconopharyngine; 16-ethoxycarbonyl-18-methoxyibogamine; 16-ethoxycarbonyl-18-methoxyibogaine; 16-ethoxycarbonyl-18-methoxyibogaline; 16-hydroxymethyl-18-methoxyibogamine; 16-hydroxymethyl-18-methoxyibogaine; 16-hydroxymethyl-18-methoxyibogaline; 18-benzyloxycoronaridine; 18-benzyloxyvoacangine; 18-benzyloxyconopharyngine; 16-ethoxycarbonyl-18-benzyloxyibogamine; 16-ethoxycarbonyl-18-benzyloxyibogaine; 16-ethoxycarbonyl-18-benzyloxyibogaine; 18-hydroxycoronaridine laurate; 18-hydroxyvoacangine laurate; 16-ethoxycarbonyl-18-hydroxyibogamine laurate; 16-ethoxycarbonyl-18-hydroxyibogaine laurate; 16-ethoxycarbonyl-18-hydroxyibogaline laurate; 18-hydroxycoronaridine acetate; 18-hydroxycoronaridine citrate; 18-hydroxycoronaridine tartrate; 18-hydroxyvoacangine acetate; 18-hydroxyvoacangine citrate; 18-hydroxyvoacangine tartrate; 18-hydroxyconopharyngine acetate; 18-hydroxyconopharyngine citrate; 18-hydroxyconopharyngine tartrate; 16-ethoxycarbonyl-18-hydroxyibogamine acetate; 16-ethoxycarbonyl-18-hydroxyibogamine citrate; 16-ethoxycarbonyl-18-hydroxyibogamine tartrate; 16-ethoxycarbonyl-18-hydroxyibogaine acetate; 16-ethoxycarbonyl-18-hydroxyibogaine citrate; 16-ethoxycarbonyl-18-hydroxyibogaine tartrate; 16-ethoxycarbonyl-18-hydroxyibogaline acetate; 16-ethoxycarbonyl-18-hydroxyibogaline citrate; 16-ethoxycarbonyl-18-hydroxyibogaline tartrate; 18-hydroxycoronaridine methoxyethoxymethyl ether; 18-hydroxyvoacangine-methoxyethoxy-methyl ether; 18-hydroxyconopharyngine-methoxy-ethoxy-methyl ether; 16-ethoxycarbonyl-18-hydroxyibogamine-methoxy-ethoxy-methyl ether; 16-ethoxycarbonyl-18-hydroxyibogaine-methoxy-ethoxy-methyl ether; 16-ethoxycarbonyl-18-hydroxyibogaline-methoxy-ethoxy-methyl ether; and pharmaceutically acceptable acids, bases and salts thereof. As used herein, pharmacologically acceptable acids, bases and salts are those acids, bases and salts that are non-toxic for use in human subjects. Toxicity is measured by those means established by those skilled in the art and may include local toxicity in a composition for local use, or systemic toxicity for systemic use, respectively. These compounds are listed for illustrative purposes only, and are not intended to be all encompassing of every α3β4 nicotinic receptor antagonist that can be used within the context of the present invention, and are in no way intended to limit the scope of the present invention. For instance, harmaline, ibogaine or its congeners, derivatives or metabolites, and other iboga alkaloids and their derivatives may prove suitable as α3β4-antagonists for the present invention.

Suitable opioid agonists for the invention include: alfenanil; allylprodine; alphaprodine; anileridine; fentanyl; sufentanil; carfentanil; lofentanil; cyclazocine; morphine; benzylmorphine; desomorphine; normorphine; dextromoramide; benzitramide; clonitazene; codeine; dihydrocodeine; levorphanol; oxycodone; oxycodone; propoxyphene; meperidine; methadone; normethadone; meptazinol; nicomorphine; LAAM; pentazocine, cyclozine, remifentanil, heroin, morphine-6-glucuronide (“M6G”); nalbuphine; buprenorphine; butorphanol; meptazinol; dezocine; diampromide; pethidine; hydromorphone; diamorphine; dihydromorphine; dimenoxadol; piritramide; nicomorphine; tilidine; tramadol; opium; beta-endorphin; met-enkaphalin; DAGO; delta-enkephalin; dynorphin A; SKF-10,047; peptide F; BAM12P; Leu-enkephalin; N-alpha-acetylmethadone; dihydromorphine; etorphine; oxymorphone; and pharmaceutically acceptable acids, bases and salts thereof. The term “opioid agonist” here is meant to mean any drug, molecule or compound that binds to and activates an opioid receptor. As an example, the mu-agonist and kappa antagonist buprenorphine, which is generally referred to as either a “partial opioid agonist” or a “mixed opioid agonist/antagonist,” is defined in this paragraph as an opioid agonist because it meets the definition of any drug, molecule or compound that binds to and activates an opioid receptor. These opioid drugs are listed for illustrative purposes only, and are not intended to be all encompassing of every opioid agonist analgesic that can be used within the context of the present invention, and are in no way intended to limit the scope of the present invention.

Factors to be taken into consideration in formulating or compounding the present invention are the elimination half-lives, volumes of distribution, affinity for target receptors, relative bioavailabilities with different routes of administration, pKₐ (H⁺dissociation constant or constants), and metabolism of the component drug compounds, etc. Also important are potential toxic effects of the component drug compounds. Taking these factors into consideration, and in light of the present invention, those skilled in the art will be able to formulate the invention based on the usual and routine laboratory and clinical testing that must be done on all drug products developed in the United States. For example, ibogaine has been shown to cause tremors in human and animal subjects alike, and at certain doses has been shown to be neurotoxic, especially on Purkinje nerve cells. 18-MC has been shown to be devoid of these undesirable characteristics. Therefore, it would be logical and consistent with the present invention to combine 18-MC in a single composition with an opioid agonist analgesic preferably over combining ibogaine with the opioid agonist. Such routine trials will confirm the optimal α3β4 antagonist/opioid agonist combination. For example, the percent “first pass metabolism” of 18-MC may be determined, and this will aid in determining the optimal amount of 18-MC in an orally administered embodiment of the invention as compared to an embodiment intended for parenteral use where the alimentary track is initially bypassed.

By way of example only, a typical course of events of bringing a drug to human use is first to test in the drug in animals such as rats. For a variety of reasons, a drug in development may be administered in a form that is most convenient for experimental methods in animals that is not the intended end use for humans. One such example is to inject a drug into the peritoneal space. Such intraperitoneal access is used, for example, in dialysis for treatment of kidney failure. It is used in rat experiments as a means of systemic administration because of relative ease of administration in these animals. Eventually, a conversion must be calculated, based on laboratory and clinical testing typical of all drug development in the United States, to develop an optimal dose for the route of administration that will eventually be used in a human, whether it is by enteral or parenteral route. These methods are established and well known to those skilled in the art.

### EXAMPLE 1

Morphine 10 and 18-MC are combined with a suitable pharmacological carrier in a single pharmaceutical composition.

### EXAMPLE 2

Oxycodone and 18-MC are combined with a suitable pharmacological carrier in a single pharmaceutical composition.

### EXAMPLE 3

Oxycodone is combined with dextromethorphan and 18-MC with a suitable pharmacological carrier as a single pharmaceutical composition.

### EXAMPLE 4

Hydrocodone is combined with mecamylamine and dextromethorphan with a suitable pharmacological carrier as a single pharmaceutical composition.

### EXAMPLE 5

Oxycodone is combined with nalmefene and 18-MC with a suitable pharmacological carrier as a single pharmaceutical composition.

### EXAMPLE 6

Oxycodone is combined with dextromethorphan, mecamylamine and nalmefene with a suitable pharmacological carrier as a single pharmaceutical composition.

Combining multiple α3β4 antagonists in a single pharmaceutical composition is not arbitrary or random. It has been shown that dextromethorphan, which is also a NMDA receptor antagonist, when administered in combination with 18-MC, yields effects involving the “wanting center” of the brain to a greater extent than can be attributed to the simple additive effects of dextromethorphan and 18-MC on the wanting center. Therefore, there appears to be a synergistic effect when 18-MC and dextromethorphan are administered together. Nalmefene, in ultra low doses may potentiate the analgesic effect of the combined opioid agonist analgesic, allowing lower doses of the opioid to be administered for a given dose to produce a certain analgesic effect. Thus, an opioid is administered at lesser dose for any licit use, while at the same time not producing a psychological wanting of the opioid for other than any licit use in this example.

The present invention also encompasses the combined administration in a single composition of an opioid agonist analgesic, a α3β4 nicotinic receptor antagonist, and an NMDA receptor antagonist. Here, NMDA receptor is meant to mean N-methyl-D-aspartate receptor that binds glycine or phenylcyclidine. There may be beneficial effects attributed to blocking the NMDA receptor that complement the beneficial effects of blocking the α3β4 receptor, when administered together with an opioid agonist analgesic. Such beneficial effects are explained separately in the prior art.

### EXAMPLE 7

Hydrocodone is combined in a single pharmaceutical composition with a α3β4 nicotinic receptor antagonist and a non-opioid analgesic such as a non-steroidal anti-inflammatory drug (“NSAID”) such as aspirin, ibuprofen, naproxen, etc., or with acetaminophen (Tylenol®), with a suitable pharmacological carrier. Other NSAID's that may be used consistent with the present invention include, but are not limited to diclofenac, benoxaprofen, flurbiprofen, fenoprofen, flubufen, ketoprofen, ketorolac, indoprofen, piroprofen, carprofen, oxaprozin, pramoprofen, muroprofen, trioxaprofen, suprofen, aminoprofen, tiaprofenic acid, indomethacin, sulindac, tolmentin, zomepirac, tiopinac, acemetacin, fentiazac, clinanac, oxpinac, piroxicam.

### EXAMPLE 8

A pharmaceutical preparation formulated for nasal administration such that active drugs are systemically absorbed via the nasal cavity mucus membranes containing butorphanol and a suitable α3β4 nicotinic receptor antagonist. The more preferred α3β4 nicotinic receptor antagonist is one that with or without permeation enhancers is better suited for absorption across the nasal mucosa. This, in light of the present invention, is easily determined by those skilled in the art based on the chemical properties of the particular α3β4 nicotinic receptor antagonist, and which is confirmed by routine laboratory and clinical testing.

### EXAMPLE 9

A pharmaceutical preparation formulated for sublingual administration containing buprenorphine as the opioid agonist analgesic, naloxone as an opioid antagonist, and a suitable α3β4 nicotinic receptor antagonist. Here the active ingredients for any licit use are buprenorphine and the α3β4 nicotinic receptor antagonist, where naloxone is usually poorly absorbed by the sublingual route of administration. The more preferred α3β4 nicotinic receptor antagonist is one that with or without permeation enhancers is better suited for absorption under the human tongue. This, in light of the present invention, is easily determined by those skilled in the art based on the chemical properties of the particular α3β4 nicotinic receptor antagonist, and which is confirmed by routine laboratory and clinical testing. Such chemical properties may include pH of the sublingual preparation, the lipid solubility of the active drug, the ionic charge of the α3β4 nicotinic receptor antagonist, and other properties that are well described in prior art and well known to those skilled in the art.

### EXAMPLE 10

A sustained release preparation of opioid agonist analgesic formulated for prolonged systemic administration by any of a known variety of means including microencasulation, inclusion within an erodable matrix, as part of a hydrogel composition, entrapment by a polymer or mixture of polymers, organic linkage as part of a degradable polymer-drug formulation, etc., containing morphine, a suitable α3β4 nicotinic receptor antagonist, and a suitable carrier thereof.

### EXAMPLE 11

A transdermal apparatus for delivering fentanyl, similar in general concept to the Duragesic® patch (Jannsen Pharmaceuticals), except that which contains a suitable α3β4 nicotinic receptor antagonist. The more preferred α3β4 nicotinic receptor antagonist is one that with or without permeation enhancers is better suited for absorption through the human skin. This, in light of the present invention, is easily determined by those skilled in the art based on the chemical properties of the particular α3β4 nicotinic receptor antagonist, and which is confirmed by routine laboratory and clinical testing. Such chemical properties may include pH of the ingredients of the transdermal apparatus that are delivered to the surface of the skin, the lipid solubility of the active drug, the ionic charge of the α3β4 nicotinic receptor antagonist, and other properties that are well described in prior art and well known to those skilled in the art.

### EXAMPLE 12

A pharmaceutical composition consisting essentially of the components of DextroMorph™ (Algos Pharmaceuticals, now Endo Pharmaceuticals) except that a suitable α3β4 nicotinic receptor antagonist is included as an additional component. Here, dextromethorphan is included as an NMDA receptor antagonist for the purposes of decreasing tolerance built to morphine and to allow a less amount of morphine to cause a given analgesic effect. Dextromethorphan as an NMDA receptor antagonist does not decrease the wanting of, the euphoric effect of, or the rewarding effect of morphine on the pleasure reward center of the brain by the mechanism described in the present invention involving the indirect dopamine decreasing affect of blocking α3β4 nicotinic receptor from acetylcholine. Other NMDA receptor antagonists such as MK-801, dextorphan or d-methadone may also be used.

It should be pointed out that many compounds, molecules or drugs that work by attaching to a known receptor, may act at more than one kind of receptor. For instance, a given α3β4 nicotinic receptor antagonist may also block α3β4 nicotinic receptors or NMDA receptors, but to a different extent, and with different affinities than it attaches to a α3β4 nicotinic receptor. The term “α3β4 nicotinic receptor antagonist” is used herein to define any drug that attaches to and blocks a α3β4 nicotinic receptor such that acetylcholine or other activating neurotransmitter is impeded from binding to and activating the α3β4 nicotinic receptor. The present patent may therefore include other receptor blockers except those that are duplicative of the present invention by way of inherency in the prior art. For example, dextromethorphan is both an α3β4 nicotinic receptor blocker as well as a NMDA receptor blocker. Where the present patent cannot claim dextromethorphan and opioid agonist as a composition due to inherency of a prior art patent (in compositions where the opioid agonist is similar in structure to morphine—see below), the present patent claims the use of dextromethorphan to decrease wanting of the opioid agonist that is distinct from its use of decreasing tolerance to, or for increasing the relative potency of, the opioid agonist which is claimed in prior art to be due to dextromethorphan's NMDA receptor blocking ability.

If the NMDA receptor antagonist is dextromethorphan (e.g. DextroMorph™), and the α3β4 nicotinic receptor antagonist here is 18-MC or mecamylamine, this will result in a decrease wanting for the morphine to an effect much greater than could possibly be effected by dextromethorphan without 18-MC or mecamylamine, respectively.

DextroMorph™ is purported to treat opioid tolerance and to decrease the amount of morphine needed for a given analgesic effect, and is not purported to decrease the wanting, of morphine. In fact, dextromethorphan as a NMDA receptor antagonist is thought to be effective in preventing tolerance and enhancing analgesia only with opioids similar in structure to morphine, and has been shown not to have such significant effects with opioid agonist analgesics of dissimilar structures. However, in the present invention, when dextromethorphan is considered as an α3β4 nicotinic receptor antagonist, it is expected that the effects of dextromethorphan would be similar on acetylcholine blockade at the α3β4 nicotinic receptor antagonist regardless of the chemical structure of the opioid agonist analgesic. Therefore, one cannot correctly argue that an opioid agonist with a chemical structure not similar to morphine, when combined with dextromethorphan for decreasing the wanting of the opioid agonist analgesic that is dissimilar to morphine, is inherent in an invention that broadly claims the combination of any opioid agonist analgesic and dextromethorphan as an NMDA receptor antagonist for the purpose of decreasing tolerance to, and decreasing the amount necessary for, the (dissimilar) opioid agonist analgesic. In this example being described, the α3β4 nicotinic receptor antagonist is added as an additional component to the NMDA receptor antagonist dextromethorphan.

### EXAMPLE 13

A pharmaceutical composition consisting essentially of the components of OxyTrex™ (Pain Theraeutics, Inc.) except that a suitable α3β4 nicotinic receptor antagonist is included as an additional component. Here, naltrexone is combined with oxycodone in a single pharmaceutical composition. The naltrexone, an opioid antagonist, is included so to act on opioid receptors such that at very low doses of naltrexone it potentiates, or at least does not antagonize, the effects at opioid receptors of the opioid agonist analgesic oxycodone, but at higher dose of naltrexone will effective block opioid receptors such that the opioid agonist effect of the oxycodone will be effectively antagonized. Neither of the active drugs of OxyTrex™ acts on nicotinic receptors to antagonize binding of acetylcholine at the receptor, which indirectly results in decreased dopaminergic effects within the pleasure-reward center of the brain.

### EXAMPLE 14

A pharmaceutical composition consisting essentially of the components of MorViva™ (Pain Therapeutics, Inc.) except that a suitable α3β4 nicotinic receptor antagonist is included as an additional component. Here, naltrexone is combined with morphine in a single pharmaceutical composition. The naltrexone, an opioid antagonist, is included so to act on opioid receptors such that at very low doses of naltrexone it potentiates, or at least does not antagonize, the effects at opioid receptors of the opioid agonist analgesic oxycodone, and the naltrexone also purportedly prevents the build up of tolerance to the morphine. Neither of the active drugs of MorViva™ acts on nicotinic receptors to antagonize binding of acetylcholine at the receptor, which indirectly results in decreased dopaminergic effects within the pleasure-reward center of the brain.

### EXAMPLE 15

A pharmaceutical composition consisting essentially of the components of OxyContin® (Purdue Pharma, LLP) except that a suitable α3β4 nicotinic receptor antagonist is included as an additional component. Here, oxycodone is released in a more prolonged or sustained release formulation for oral administration and enteric absorption. OxyContin® when crushed, loses its sustained release characteristics, such that absorption is fast. This method of crushing OxyContin® tablets has been used illicitly by those seeking euphoric effects rather than analgesia or any licit use, and the crushed tablets are then ingested orally (per os), injected intravenously, or “snorted” (insufflation) through the nares (nostrils) for absorption through nasal mucosa. Including an effective amount of a α3β4 nicotinic receptor antagonist, as for example 18-MC, would tend to negate the euphoric affects. Thus, a human seeking effects of oxycodone other than for any licit use would be less motivated to crush an “OxyContin®-18-MC-containing” tablet. This would be of great societal importance.

### EXAMPLE 16

A pharmaceutical preparation formulated for intravenous, intramuscular or subcutaneous administration containing meperidine as the active opioid agonist analgesic ingredient, which also contains a suitable amount of a α3β4 nicotinic receptor antagonist to effectively diminish the increase in dopamine in the pleasure-reward center of the brain that is associated with meperidine administration.

### EXAMPLE 17

A pharmaceutical composition formulated for oral use consisting essentially of an opioid agonist, wherein an analgesically effective amount of an orally active opioid agonist is combined with an opioid antagonist into an oral dosage form which would require at least a two-step extraction process to be separated from the opioid agonist, the amount of antagonist extracted being sufficient to counteract the opioid agonist effects if extracted together with the opioid agonist and administered parenterally, which also contains α3β4 nicotinic receptor antagonist, preferably with the α3β4 nicotinic receptor antagonist contained within the same extraction compartment as the opioid agonist analgesic. By more specific example, the opioid agonist analgesic is hydromorphone, the opioid antagonist is nalmefene hydrochloride and the α3β4 nicotinic receptor antagonist is 18-MC.

Nalmefene is more preferred as opioid antagonist than naltrexone when combined in a single composition with an opioid agonist analgesic because nalmefene has more simple pharmacokinetics in that naltrexone is metabolized in humans to 6-beta-naltrexol, which is a more potent and longer lasting opioid antagonist than is its parent compound naltrexone. Thus, with naltrexone administration, there will be two circulating effective opioid antagonists with different binding affinities and different termination half-lives, occurring at constantly changing ratios of one to the other (the parent naltrexone, and the metabolite 6-beta-naltrexol). On the other hand, nalmefene has no appreciably active metabolites in humans, therefore opioid blockade due to circulating concentrations of active antagonist is much more easy to predict with nalmefene than with naltrexone.

### EXAMPLE 18

A pharmaceutical composition consisting essentially of the components of Lortab® (UCB Pharma, Inc.) except that a suitable α3β4 nicotinic receptor antagonist is included as an additional component.

### EXAMPLE 19

A pharmaceutical composition consisting essentially of the components of Vicoprofen® (Knoll Laboratories) except that 18-MC is included as an additional component.

### EXAMPLE 20

A pharmaceutical composition consisting essentially of the components of Percocet® (Endo Pharmaceuticals, Inc.) except that mecamylamine is included as an additional component.

### EXAMPLE 21

A pharmaceutical composition consisting essentially of the components of Vicodin® (Knoll Laboratories) except that a suitable α3β4 nicotinic receptor antagonist is included as an additional component.

### EXAMPLE 22

The opioid agonist analgesic hydrocodone is included in a pharmaceutical tablet composition with an opioid antagonist where the opioid antagonist is separately encapsulated with a coating that is generally resistant to digestion or degradation by non-traumatic transfer through the alimentary or gastrointestinal track. Here, “non-traumatic” is meant to mean the natural passage through the alimentary system that would not cause a physical crushing of such a small coated particle as is the encapsulated opioid antagonist. When ingested orally, the opioid antagonist is not released from encapsulation, preventing its systemic action on opioid antagonists. However, if the tablet composition is physically crushed by traumatic means prior to ingestion or administration, whether it is by enteral or parenteral administration, the opioid antagonist will be released from encapsulation, thereby making it available for systemic action to antagonize the opioid agonist analgesic included within the tablet composition at opioid receptors. The encapsulation may be accomplished by any of a known number of means described in the prior art, such as by coating with a polymer that is resistant to the digestive process within the gastrointestinal track. Included within the tablet would be a α3β4 nicotinic receptor antagonist, such as 18-MC. The 18-MC could be contained as an admixture with the hydrocodone, or within the encapsulation with the opioid antagonist, or with both the hydrocodone and within the encapsulation with the opioid antagonist. For completion's sake of this example, the opioid antagonist is naltrexone.

Though many examples are presented herein that embody the present invention, they are for illustrative purposes only and are not intended to limit the scope of the present invention. Further, though the mechanisms of action presented herein are stated in good faith as those postulated to be responsible for the working of the present invention, the invention is not defined solely by the theory of action, but rather also by the composition or compositions taught herein, and therefore it is claimed by letters patent the compositions and methods written below, regardless of any subsequent work that might alter the body of knowledge upon which the postulated mechanisms of action may now rely.

The more preferred α3β4 nicotinic receptor antagonists for the present invention are those that are most selective for the α3β4 nicotinic receptor, so as to not cause unwanted other effects by binding to and either activating or blocking other receptors. In this respect, 18-MC is predicted to have a much wider therapeutic window than ibogaine, for example, which blocks not only α3β4 nicotinic receptors but NMDA and sigma-2 receptors, and sodium channels and the 5-HT transporter as well. Also, because the essence of the present invention is the simultaneous administration of both an opioid agonist analgesic and an α3β4 nicotinic receptor antagonist, the present invention cannot possibly be considered without taking into account what effects the α3β4 nicotinic receptor antagonist may have on opioid receptors. For example, it has been demonstrated that both ibogaine and 18-MC have similar affinities as one another for kappa opioid receptors. When determining the ideal α3β4 nicotinic receptor antagonist for the present invention, one would preferably select an α3β4 nicotinic receptor antagonist that did not block mu opioid receptors so as to allow maximum analgesia in the presence of decreased wanting of the drug. Because stereochemistry of α3β4 nicotinic receptor antagonists has been shown to influence affinities at mu and delta receptors (Bioorganic & Medicinal Chemistry Letters 10 (2000) 473-476, p.474-third paragraph), the present invention cannot be reasonably considered “without regard to the stereochemistry.” In fact, one would expect the (−)-enantiomer of 18-MC to be the more preferred stereoisomer of 18-MC for the present invention because (−)-18-methoxycoronaridine has a 10-fold lesser affinity to bind (and presumably block) mu opioid receptor than does the (+)-enantiomer.

I claim:

1.) An analgesic composition that comprises an effective amount of an opioid agonist, an α3β4 nicotinic receptor antagonist and a suitable carrier thereof.

2.) A method for treating pain or diarrhea in a human, wherein the method comprises administering to said human a single pharmaceutical composition containing an opioid agonist analgesic and an α3β4 nicotinic receptor antagonist.

3.) A method for any licit use of an opioid agonist in a human that decreases the wanting of said opioid agonist by said human other than for said any licit use, wherein the method comprises administering to said human said opioid agonist simultaneously with an α3β4 nicotinic receptor antagonist.

4.) A pharmaceutical composition comprising an opioid agonist analgesic, an α3β4 nicotinic receptor antagonist, and a suitable carrier thereof.

5.) A pharmaceutical composition comprising an opioid agonist analgesic, an α3β4 nicotinic receptor antagonist, a non-steroidal anti-inflammatory drug, and a suitable carrier thereof.

6.) A pharmaceutical composition comprising an opioid agonist analgesic, an α3β4 nicotinic receptor antagonist, a NMDA-receptor antagonist, and a suitable carrier thereof.

7.) A pharmaceutical composition comprising an opioid agonist analgesic, an α3β4 nicotinic receptor antagonist, a NMDA-receptor antagonist, a non-steroidal anti-inflammatory drug, and a suitable carrier thereof.

8.) A pharmaceutical composition comprising an opioid agonist analgesic, an α3β4 nicotinic receptor antagonist, an opioid antagonist, and a suitable carrier thereof.

9.) A pharmaceutical composition comprising an opioid agonist analgesic, an α3β4 nicotinic receptor antagonist, a non-steroidal anti-inflammatory drug, an opioid antagonist, and a suitable carrier thereof.

10.) A pharmaceutical composition comprising an opioid agonist analgesic, an α3β4 nicotinic receptor antagonist, a NMDA-receptor antagonist, an opioid antagonist, and a suitable carrier thereof.

11.) A pharmaceutical composition comprising an opioid agonist analgesic, an α3β4 nicotinic receptor antagonist, a NMDA-receptor antagonist, an opioid antagonist, a non-steroidal inflammatory drug, and a suitable carrier thereof.

12.) The claim of claim 4 above where the α3β4 nicotinic receptor antagonist is any from the group of ibogaine, 18-methoxycoronaridine and mecamylamine, and the opioid agonist analgesic is any from the group of alfenanil; allylprodine; alphaprodine; anileridine; fentanyl; sufentanil; carfentanil; lofentanil; cyclazocine; morphine; benzylmorphine; desomorphine; normorphine; dextromoramide; benzitramide; clonitazene; codeine; dihydrocodeine; levorphanol; oxycodone; oxycodone; propoxyphene; meperidine; methadone; normethadone; meptazinol; nicomorphine; LAAM; pentazocine, cyclozine, remifentanil, heroin, morphine-6-glucuronide (“M6G”); nalbuphine; buprenorphine; butorphanol; meptazinol; dezocine; diampromide; pethidine; hydromorphone; diamorphine; dihydromorphine; dimenoxadol; piritramide; nicomorphine; tilidine; tramadol; opium; beta-endorphin; met-enkaphalin; DAGO; delta-enkephalin; dynorphin A; SKF-10,047; peptide F; BAM12P; Leuenkephalin; N-alpha-acetylmethadone; dihydromorphine; etorphine; oxymorphone.

13.) The claim of claim 5 above where the non-steroidal anti-inflammatory drug is any from the group of aspirin, ibuprofen, naproxen, diclofenac, benoxaprofen, flurbiprofen, fenoprofen, flubufen, ketoprofen, ketorolac, indoprofen, piroprofen, carprofen, oxaprozin, pramoprofen, muroprofen, trioxaprofen, suprofen, aminoprofen, tiaprofenic acid, indomethacin, sulindac, tolmentin, zomepirac, tiopinac, acemetacin, fentiazac, clinanac, oxpinac, piroxicam.

14.) The claim of claim 6 above where the NMDA-receptor antagonist on any from the group of dextromethorphan, MK-801, dextorphan or dmethadone.

15.) The method of claim 2 above where said single pharmaceutical composition comprises a sustained release preparation of opioid agonist analgesic.

16.) An analgesic composition that comprises an effective amount of an opioid agonist, and two or more α3β4 nicotinic receptor antagonists that act synergistically with one another, and a suitable carrier thereof.

17.) The claim of claim 3 above, where the α3β4 nicotinic receptor antagonist any from the group of dextromethorphan, dextorphan, 18-MC and mecamylamine and ibogaine.

18.) The claim of claim 1 where the α3β4 nicotinic receptor antagonist is (−)-18-methoxycoronaridine.
